(19) United States
(12) Patent Application Publication
(10) Pub. No.: US 2009/0252698 A1
(43) Pub. Date: Oct. 8, 2009

(54) EMULSION COMPOSITIONS FOR EYELASHES

(75) Inventors: Sue FENG, Edison, NJ (US); Kavita PATEL, North Brunswick, NJ (US); Lilavati PATEL, Edison, NJ (US)

Correspondence Address: OBLON, SPIVAK, MCCLELLAND MAIER & NEUSTADT, P.C., 1940 DUKE STREET, ALEXANDRIA, VA, 22314, US

(73) Assignee: L'Oreal S.A., Paris (FR)

(21) Appl. No.: 12/416,282

(22) Filed: Apr. 1, 2009

Related U.S. Application Data
(60) Provisional application No. 61/041,951, filed on Apr. 3, 2008.

(51) Int. Cl. A61K 8/892; A61Q 1/10

(52) U.S. Cl. 424/70.7; 424/70.12

(57) ABSTRACT

The invention relates to emulsion compositions for application to eyelashes containing at least one surfactant comprising silicone, hydrocarbon and glycerol group(s), as well as to methods of applying such compositions to eyelashes and kits containing such compositions.

### REFERENCE TO PRIOR APPLICATIONS

This application claims priority to U.S. provisional application 61/041,951, filed Apr. 3, 2008, incorporated herein by reference in its entirety.

### FIELD OF THE INVENTION

The present invention generally relates to emulsion compositions comprising a surfactant comprising silicone, hydrocarbon and glycerol group(s) as well as to methods of applying such compositions to eyelashes and kits comprising such compositions. Such emulsion compositions possess improved properties and characteristics such as, for example, improved deposition characteristics upon application (for example, improved speed of deposition), improved volumizing characteristics upon application and/or improved lengthening characteristics upon application.

### DISCUSSION OF THE BACKGROUND

Traditional compositions for application to eyelashes (mascaras, topcoats, basecoats) can be characterized as creamy emulsion compositions. Such traditional compositions contain an oil phase and a water phase, and contain significant amounts of wax. In the past, traditional basecoat compositions lack transparency owing to their high wax content: that is, after application to eyelashes and drying, the basecoat compositions have a whitish coloration. Such white basecoat layers on eyelashes can adversely affect the visual appearance of any topcoat or mascara which is applied to the basecoat.

Also, traditional compositions for application to eyelashes can have poor deposition characteristics upon application. For example, such compositions, when applied to eyelashes, do not adhere well to eyelashes, do not distribute evenly on the eyelashes and/or clump upon application. The result of such problems is that applying such compositions to eyelashes can be slow and uneven, taking multiple applications to achieve a desired look. Thus, there is a need for improved emulsion compositions for application to eyelashes.

Accordingly, one aspect of the present invention is a care and/or makeup and/or treatment composition for eyelashes which is able to address or overcome the aforementioned problems with traditional compositions.

### SUMMARY OF THE INVENTION

The present invention relates to emulsion compositions for application to eyelashes comprising at least one surfactant comprising silicone, hydrocarbon and glycerol group(s). Such a composition could be, for example, a basecoat upon which a mascara could be applied, a topcoat which could be applied on a mascara, or a mascara.

The present invention also relates to compositions for eyelashes such as mascaras, topcoats and basecoats comprising at least one surfactant comprising silicone, hydrocarbon and glycerol group(s), and at least one surfactant comprising silicone and glycerol group(s) but which lacks hydrocarbon groups. Such a composition could also include at least one co-emulsifier comprising silicone, hydrocarbon and polyether (for example, polyethylene glycol and/or polypropylene glycol) group(s).

The present invention further relates to methods of improving adhesion to and/or distribution on eyelashes of a composition for application to eyelashes comprising adding at least one surfactant comprising silicone, hydrocarbon and glycerol group(s) to the composition. Such a method could also include adding at least one surfactant comprising silicone and glycerol group(s) but which lacks hydrocarbon groups and/or at least one co-emulsifier comprising silicone, hydrocarbon and polyether (for example, polyethylene glycol and/or polypropylene glycol) group(s) to the composition.

The present invention further relates to methods of lengthening and/or volumizing eyelashes comprising applying to the eyelashes an eyelash lengthening and/or volumizing effective amount of a composition of the present invention.

The present invention also relates to methods of treating, caring for, making up or enhancing the appearance of eyelashes comprising applying compositions of the present invention to the eyelashes in an amount sufficient to treat, care for, make-up and/or enhance the appearance of the eyelashes.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Cosmetically acceptable” means that the item in question is compatible with eyelashes.

“Transfer resistance” as used herein refers to the quality exhibited by compositions that are not readily removed by contact with another material, such as, for example, an item of clothing or the skin. Transfer resistance may be evaluated by any method known in the art for evaluating such. For example, transfer resistance of a composition may be evaluated by a modified “kiss” test. The modified “kiss” test may involve application of the composition to human eyelashes followed by “kissing” a material, for example, a sheet of paper, after expiration of a certain amount of time following application, such as 2 minutes after application. Similarly, transfer resistance of a composition may be evaluated by the amount of product transferred from a wearer to any other substrate, such as transfer from eyelashes of an individual to clothing after the expiration of a certain amount of time following application. The amount of composition transferred to the substrate (e.g., clothing or paper) may then be evaluated and compared. For example, a composition may be transfer resistant if a majority of the product is left on the eyelashes. Further, the amount transferred may be compared with that transferred by other compositions, such as commercially available compositions.

“Long wear” compositions as used herein, refers to compositions where at least one property chosen from consistency, texture, and color remains the same as at the time of application, as viewed by the naked eye, after an extended period of time, such as, for example, 1 hour, 2 hours, and further such as 8 hours. Long wear properties may be evaluated by any method known in the art for evaluating such properties. For example, long wear may be evaluated by a test involving the application of a composition to human eyelashes and evaluating the consistency, texture and color of the composition after an extended period of time. For example, the consistency, texture and color of a mascara may be evaluated immediately following application and these characteristics may then be re-evaluated and compared after an individual has worn the lip composition for a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions.

“Tackiness” as used herein refers to measuring the maximum tensile force, Fₘₐₓ, required while separating two surfaces. Depending on the application envisaged and the formulation being designed, the desirable value for Fₘₐₓ may vary. In some embodiments, the substantially non-tacky compositions have a Fₘₐₓ of less than about 4 Newton (N), less than about 1 N, less than about 0.5 N, less than about 0.3 N, less than about 0.2 N or less than 0.1 N. One of ordinary skill in the art can determine the Fₘₐₓ of the composition by, for example, determining the maximum force of traction, measured with an extensiometer of the LLOYD model LR5K type, needed to detach two surfaces.

For example, two 38 mm² surfaces, A and B, which are solid, rigid, inert, and non-absorbing, are mounted on movable mounts, facing each other. The surfaces may be movable either toward or away from each other, or one may move surface A independently from surface B or vice versa. Prior to insertion into the extensiometer, surface A is coated with the composition to be measured, which may be dissolved in a solvent such as aqueous, hydroalcoholic, hydrocarbon, silicone, and alcoholic solvents in a concentration of from about 10 to about 30%, preferably 20%, the surface A is coated in a thickness of from 1 to 10 mil, preferably 1 mil, and the surface is dried for 24 hours at room temperature, e.g., 22 to 25° C., at a relative humidity of about 50%. Once inserted in the extensiometer, surface A is subjected for 20 seconds to a compression force of 3 N against surface B and then subjected for 30 seconds to tensile force at a rate of 20 mm/minute. The amount of force, Fₘₐₓ, needed to obtain initial separation is then noted. A mean Fₘₐₓ is determined by carrying out the procedure with multiple pairs, preferably at least six pairs, of surface A and surface B.

The compositions, methods and kits of the present invention can comprise, consist of, or consist essentially of the essential elements and limitations of the invention described herein, as well as any additional or optional ingredients, components, or limitations described herein or any otherwise useful ingredient found in personal care compositions intended for application to eyelashes.

The composition of the present invention may be in any suitable emulsion form for use on eyelashes. Preferably, the emulsion compositions of the present invention possess an exterior aqueous phase (for example, an oil-in-water emulsion, a water-in-oil-in water triple emulsion, etc.). Most preferably, the emulsion compositions of the present invention are oil-in-water emulsions. The compositions of the present invention can be mascaras. Generally speaking, mascaras contain colorants such as pigments. The compositions of the present invention can be used as a basecoat and/or topcoat for application beneath and/or onto other products applied to eyelashes.

The emulsion compositions of the present invention preferably contain from about 5% to about 95% by weight of the total weight of the composition of water, more preferably from about 15% to about 80% of the total weight of the composition, and most preferably from about 20% to about 70%, including all ranges and subranges therebetween.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation. The stability is further tested by repeating the 8-week test at 40° C., 37° C., 45° C., 50° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

The term “transparent or translucent” according to the present invention is understood to mean compositions exhibiting a turbidity, measured according to the method described below, of less than 800 NTU (Nephelometric Turbidity Units) and preferably of less than 500 NTU. The turbidity is measured using a model 2100 P turbidimeter from Hach at ambient temperature (20 to 25° C.). The tubes used for the measurement are referenced AR 397 A cat 24347-06. The device is calibrated using formazine suspensions with different concentrations.

Surfactant comprising silicone, hydrocarbon and glycerol group(s).

According to the present invention, compositions comprising at least one surfactant comprising silicone, hydrocarbon and glycerol group(s) are provided. In accordance with the present invention, the surfactant can be any suitable, branched surfactant containing all three required groups. A “branched surfactant” as used herein refers to a compound comprising (a) a backbone comprising silicone; and (b) at least one side chain comprising silicon (such as, for example, a siloxane group).

According to the present invention, the surfactant comprises a silicone backbone comprising at least two silicon based groups of the formula R₂SiO, where R₂ represents a linear or branched, saturated or unsaturated, C₁ to C₁₀ hydrocarbon-based group. Preferably, R₂ is an unsubstituted C₁ to C₃ alkyl group (methyl, ethyl, propyl), most preferably a methyl group. The silicone backbone contains multiple repeating units of the silicon based group such as, for example, between 2 and 1000, preferably between 10 and 500, more preferably between 15 and 400, and more preferably between 20 and 350 repeating units, including all ranges and subranges therebetween. Preferably, the silicone backbone is a polyalkylsiloxy backbone. According to preferred embodiments, the silicone backbone is a polydimethylsiloxy backbone.

According to the present invention, the surfactant comprises at least one hydrocarbon group. The hydrocarbon group comprises a linear or branched, saturated or unsaturated, substituted or unsubstituted group which contains at least 6 carbon atoms. Preferably, the hydrocarbon group contains 8 to 24 carbon atoms, more preferably 9 to 18 carbon atoms, and most preferably 10 to 16 carbon atoms, including all ranges and subranges therebetween. According to preferred embodiments, the hydrocarbon group is a linear, saturated, unsubstituted group, most preferably containing 10 to 16 carbon atoms. The most preferred hydrocarbon group is a lauryl group.

According to the present invention, the surfactant comprises at least one glycerol group. Preferably, the surfactant contains two or more glycerol groups and, thus, is polyglycerylated. Most preferably, the surfactant contains 3 glycerol groups.

According to particularly preferred embodiments, the surfactant comprising siloxane, hydrocarbon and glycerol group(s) is lauryl polyglyceryl-3 polydimethylsiloxyethyl such as that sold by Shin-Etsu under the trade name KF 6105.

Preferably, the amount of surfactant comprising siloxane, hydrocarbon and glycerol group(s) present in the composition preferably ranges from about 0.5% to about 5 by weight with respect to the total weight of the composition, more preferably from about 0.75% to about 4% by weight with respect to the total weight of the composition, and more preferably from about 1% to about 3% by weight with respect to the total weight of the composition, including all ranges and subranges therebetween.

Surfactant comprising silicone and glycerol group(s) but which lacks hydrocarbon groups

According to preferred embodiments of the present invention, the composition further comprises and at least one additional surfactant comprising silicone and glycerol group(s) but which lacks hydrocarbon groups. In accordance with the present invention, the surfactant can be any suitable, branched surfactant containing both required groups but which lacks a hydrocarbon group. A “branched surfactant” as used in this context is the same as above.

According to the present invention, the surfactant comprises a silicone backbone comprising at least two silicon based groups of the formula R₂SiO, where R₂ represents a linear or branched, saturated or unsaturated, C₁ to C₁₀ hydrocarbon-based group. Preferably, R₂ is an unsubstituted C₁ to C₃ alkyl group (methyl, ethyl, propyl), most preferably a methyl group. The silicone backbone contains multiple repeating units of the silicon based group such as, for example, between 2 and 1000, preferably between 10 and 500, more preferably between 15 and 400, and more preferably between 20 and 350 repeating units, including all ranges and subranges therebetween. Preferably, the silicone backbone is a polyalkylsiloxy backbone. According to preferred embodiments, the silicone backbone is a polydimethylsiloxy backbone.

According to the present invention, the surfactant comprises at least one glycerol group. Preferably, the surfactant contains two or more glycerol groups and, thus, is polyglycerylated. Most preferably, the surfactant contains 3 glycerol groups.

According to the present invention, other than R₂ in the silicone backbone which can contain up to 10 carbon atoms, the surfactant does not comprise any hydrocarbon group containing at least 6 carbon atoms.

According to particularly preferred embodiments, the surfactant comprising silicone and glycerol group(s) but lacking a hydrocarbon group is polyglyceryl-3 disiloxane dimethicone (for example, such as that sold by Shin Etsu under the trade name KF 6100) or polyglyceryl-3 polydimethylsiloxyethyl dimethicone (KF 6104).

Preferably, the amount of surfactant comprising silicone and glycerol group(s) but lacking hydrocarbon group(s) present in the composition preferably ranges from about 0.5% to about 10% by weight with respect to the total weight of the composition, more preferably from about 0.75% to about 8% by weight with respect to the total weight of the composition, and more preferably from about 1 % to about 6% by weight with respect to the total weight of the composition, including all ranges and subranges therebetween.

According to preferred embodiments, compositions of the present invention preferably contain more surfactant comprising silicone, hydrocarbon and glycerol group(s) than surfactant comprising silicone and glycerol group(s) but which lacks hydrocarbon groups. Preferably, the ratio of surfactant comprising silicone, hydrocarbon and glycerol group(s) to surfactant comprising silicone and glycerol group(s) but which lacks hydrocarbon groups is at least 2:1, at least 2.5:1, or at least 3:1, including all ranges and subranges therebetween.

Co-emulsifier comprising silicone, hydrocarbon and polyether group(s).

According to preferred embodiments of the present invention, the composition further comprises at least one co-emulsifier comprising silicone, hydrocarbon and polyether group(s). In accordance with the present invention, the surfactant can be any suitable, branched surfactant containing all three required groups. A “branched surfactant” as used in this context is the same as above.

According to the present invention, the surfactant comprises a silicone backbone comprising at least two silicon based groups of the formula R₂SiO, where R₂ represents a linear or branched, saturated or unsaturated, C₁ to C₁₀ hydrocarbon-based group. Preferably, R₂ is an unsubstituted C₁ to C₃ alkyl group (methyl, ethyl, propyl), most preferably a methyl group. The silicone backbone contains multiple repeating units of the silicon based group such as, for example, between 2 and 1000, preferably between 10 and 500, more preferably between 15 and 400, and more preferably between 20 and 350 repeating units, including all ranges and subranges therebetween. Preferably, the silicone backbone is a polyalkylsiloxy backbone. According to preferred embodiments, the silicone backbone is a polydimethylsiloxy backbone.

According to the present invention, the surfactant comprises at least one hydrocarbon group. The hydrocarbon group comprises a linear or branched, saturated or unsaturated, substituted or unsubstituted group which contains at least 6 carbon atoms. Preferably, the hydrocarbon group contains 8 to 24 carbon atoms, more preferably 9 to 18 carbon atoms, and most preferably 10 to 16 carbon atoms, including all ranges and subranges therebetween. According to preferred embodiments, the hydrocarbon group is a linear, saturated, unsubstituted group, most preferably containing 10 to 16 carbon atoms. The most preferred hydrocarbon group is a lauryl group.

According to the present invention, the surfactant comprises at least polyether group comprising oxygen and a C₁ to C₁₀ hydrocarbon-based group, preferably a C₂ to C₃ alkyl group (methyl, ethyl). Most preferably, the surfactant comprises at least one polyether group selected from the group consisting of polyethylene glycol, polypropylene glycol, and mixtures thereof. groups). The polyether group contains multiple repeating units such as, for example, between 2 and 100, preferably between 3 and 20, more preferably between 4 and 15, and more preferably between 5 and 10 repeating units, including all ranges and subranges therebetween.

Examples of suitable co-emulsifiers comprising silicone, hydrocarbon and polyether group(s) include PEG/PPG-10/3 oleyl ether dimethicone, PEG-9 polydimethylsiloxyethyl dimethicone, and lauryl PEG-9 polydimethylsiloxy dimethicone. Preferably, the co-emulsifier is lauryl PEG-9 polydimethylsiloxy dimethicone (for example, such as that sold by Shin Etsu under the trade name KF 6038).

Preferably, the amount of co-emulsifier present in the composition preferably ranges from about 0.5% to about 5 by weight with respect to the total weight of the composition, more preferably from about 0.75% to about 3% by weight with respect to the total weight of the composition, and more preferably from about 1% to about 2% by weight with respect to the total weight of the composition, including all ranges and subranges therebetween.

Additional Ingredients

The compositions of the present invention can also comprise any additive usually used in cosmetic or dermatologic compositions. For example, waxes, film forming agents, dispersants, antioxidants, oils, preserving agents, fragrances, fillers, neutralizing agents, cosmetic and dermatological active agents such as, for example, emollients, moisturizers, vitamins, anti-wrinkle agents, essential fatty acids, and sunscreens, surfactants, and mixtures thereof can be added. A non-exhaustive listing of such ingredients can be found in U.S. patent application Ser. No. 10/733,467, filed Dec. 12, 2003, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

Specific examples of additional ingredients include oils. Preferably, the compositions of the present invention further comprise at least one oil. Any oils can be used in accordance with the present invention. The oils can be volatile or non-volatile, silicone-based and/or hydrocarbon-based, etc. Thus, for example, the external oil phase may contain, independently or in combination, volatile silicone oils, non-volatile silicone oils, volatile non-silicone oils and non-volatile non-silicone oils.

In one embodiment, the compositions of the present invention are substantially free of silicone oils (i.e., contain less than about 1% of silicone oil). In another embodiment, the compositions are substantially free of non-silicone oils (i.e., contain less than about 1% of non-silicone oil). In another embodiment, the compositions are substantially free of non-volatile oils (i.e., contain less than about 1% of non-volatile oil). In yet another embodiment, the compositions are substantially free of volatile oils (i.e., contain less than about 1% of volatile oil).

The composition preferably also comprises one or more waxes. For the purposes of the present invention, a wax is a lipophilic fatty compound that is solid at room temperature (25° C.) and atmospheric pressure (760 mmHg, i.e. 101 KPa), which undergoes a reversible solid/liquid change of state, having a melting point of greater than 40° C. and further such as greater than 55° C. and which may be up to 200° C. In accordance with preferred embodiments of the present invention, the compositions comprise at least 5% wax by weight of the total weight of the composition, preferably from about 5% to about 30%, more preferably from about 7% to about 25%, and most preferably from about 10% to about 20%, including all ranges and subranges therebetween. Most preferably, the majority of wax present in the invention compositions is hydrocarbon wax(es). For example, preferably, hydrocarbon wax(es) comprise at least 51%, more preferably at least 60%, and more preferably at least 75% by weight of the total amount of wax present in the composition. It is possible that the compositions contain only hydrocarbon waxes, if desired.

For the purposes of the invention, the waxes are those generally used in cosmetics and dermatology; they are, for example, of natural origin, for instance beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methyl-siloxane esters that are solid at 40° C., for example, at above 55° C. According to the invention, the melting point values correspond to the melting peak measured by the “Differential Scanning Calorimetry” method with a temperature rise of 5 or 10° C./min.

The compositions may also optionally comprise at least one coloring agent. Suitable coloring agents include but are not limited to glitter, pigments, dyes, such as liposoluble dyes, and nacreous pigments.

“Glitter” is material which reflects flashes of light and, thus, appears to sparkle. Typically, glitter comprises a plurality of particles (i.e., pieces or fragments of a material) having a regular or irregular periphery, which reflects or refracts light. Materials useful as glitter include particles of metal (e.g., aluminum, copper, silver, gold, and brass), particles of transparent or colored, solid organic materials (e.g., polyethylene terephthalate, polymethacrylate, and polyvinylbutyral), and particles of metal coated film or paper (e.g., aluminum coated polyethylene terephthalate film). Glitter may be clear and/or be provided in a variety of colors (e.g., silver, gold, blue, red, etc.), or mixtures thereof; and may be provided in a variety of shapes (e.g., circles, squares, rectangles, triangles, diamonds, stars, symbols, alphanumerics (i.e., letters and/or numbers), or mixtures of different shapes.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0,5% to 40%, and further such as from 2% to 30%, including all ranges and subranges therebetween. In the case of certain products, the pigments, including nacreous pigments, may, for example, represent up to 50% by weight of the composition

According to preferred embodiments of the present invention, methods of applying a composition comprising at least one surfactant comprising silicone, hydrocarbon and glycerol group(s) to eyelashes are provided. According to this embodiment, the composition preferably further comprises at least one surfactant comprising silicone and glycerol group(s) but which lacks hydrocarbon groups and/or at least one co-emulsifier comprising silicone, hydrocarbon and polyether groups.

According to preferred embodiments of the present invention, methods of lengthening and/or volumizing eyelashes comprising applying to the eyelashes an eyelash lengthening and/or volumizing effective amount of a composition comprising at least one surfactant comprising silicone, hydrocarbon and glycerol group(s) to eyelashes are provided. According to this embodiment, the composition preferably further comprises at least one surfactant comprising silicone and glycerol group(s) but which lacks hydrocarbon groups and/or at least one co-emulsifier comprising silicone, hydrocarbon and polyether groups.

According to other preferred embodiments of the present invention, methods of enhancing the appearance of eyelashes comprising applying to the eyelashes a composition comprising at least one surfactant comprising silicone, hydrocarbon and glycerol group(s) to eyelashes are provided. According to this embodiment, the composition preferably further comprises at least one surfactant comprising silicone and glycerol group(s) but which lacks hydrocarbon groups and/or at least one co-emulsifier comprising silicone, hydrocarbon and polyether groups.

According to other preferred embodiments of the present invention, methods of enhancing the appearance of eyelashes comprising applying to the eyelashes a composition comprising at least one surfactant comprising silicone, hydrocarbon and glycerol group(s), and then applying to the composition a topcoat composition, are provided. According to this embodiment, the composition preferably further comprises at least one surfactant comprising silicone and glycerol group(s) but which lacks hydrocarbon groups and/or at least one co-emulsifier comprising silicone, hydrocarbon and polyether groups.

According to other preferred embodiments of the present invention, methods of enhancing the appearance of eyelashes comprising applying to the eyelashes a basecoat composition, and then applying to the basecoat composition a composition comprising at least one surfactant comprising silicone, hydrocarbon and glycerol group(s), are provided. According to this embodiment, the composition preferably further comprises at least one surfactant comprising silicone and glycerol group(s) but which lacks hydrocarbon groups and/or at least one co-emulsifier comprising silicone, hydrocarbon and polyether groups.

The present invention further relates to methods of improving adhesion to and/or distribution on eyelashes of a composition for application to eyelashes comprising adding at least one surfactant comprising silicone, hydrocarbon and glycerol group(s) to the composition.

According to still other preferred embodiments of the present invention, methods of treating, caring for, or making up eyelashes comprising applying compositions of the present invention to eyelashes in an amount sufficient to treat, care for, and/or make-up the appearance of the eyelashes are provided.

In accordance with all of the foregoing embodiments of the present invention, the compositions of the present invention may be applied to eyelashes as needed, preferably once or twice daily, more preferably once daily, and then preferably allowed to dry before subjecting to contact such as with clothing or other objects.

The present invention also envisages kits and/or prepackaged materials suitable for consumer use containing one or more compositions according to the description herein (for example, kits containing (1) a composition for application to eyelashes such as a mascara; and (2) a basecoat and/or topcoat). According to particularly preferred embodiments, kits comprising (a) a basecoat composition comprising at least one surfactant comprising silicone, hydrocarbon and glycerol group(s); and (b) a topcoat which is a mascara composition are provided. Such kits may also include other compositions or components such as, for example, a cosmetic removing composition, instructions for applying or using the compositions in the kit, cosmetic application devices (for example, a mascara brush), etc.

The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLE 1

Mascara

| Phase | INCI Name | (%) |
|---|---|---|
| A1 | Water | 54.47 |
| | Preservative | 0.22 |
| | Hydroxyethylcellulose | 0.20 |
| | Glyceryl Stearate | 4.00 |
| | Simethicone | 0.12 |
| | KF 6100 | 6.00 |
| B1 | KF6105 | 3.00 |
| | KF 6038 | 2.00 |
| | Waxes | 13.25 |
| | Olive Esther | 6.30 |
| | Preservatives | 0.30 |
| B2 | Water | 1.52 |
| | Acacia Senegal | 1.52 |
| C | Diatosol SJ | 5.00 |
| D | Dimethicone Copolyl | 0.20 |
| E | Water | 0.90 |
| | Panthenol | 1.00 |
| | TOTAL | 100.00 |

Water, methylparaben and hydroxyethylcellulose from Phase A were added to the Main Kettle and heated to 85° C., mixing until uniform using a homogenizer. Once the temperature reached 85° C., glyceryl stearate was gradually added, followed by simethicone and KF 6100 at 5 minute intervals. The composition was mixed until uniform.

Phase B1 was then mixed in a side kettle, and heated to 90° C. to melt all waxes. At 90° C., Phase B2 was added to Phase B1 using a lightening mixer until uniform. Once uniform, Phase B1 was added to Phase A in the Main Kettle and emulsified for 20 minutes. Then, the composition was cooled to 45° C. with sweep mixing at which time Phase C was added with sweep mixing. Then, the composition was cooled to 35° C. at which time Phase D was added with sweep mixing. Then, pre-mixed Phase E was added with sweep mixing. The composition was then cooled to 25° C.

What is claimed is:

1. An emulsion having an outer aqueous phase comprising:
(a) at least one branched surfactant comprising silicone, hydrocarbon and glycerol group(s);
(b) at least one branched surfactant comprising silicone and glycerol group(s) but which lacks hydrocarbon groups; and
(c) at least one branched co-emulsifier comprising silicone, hydrocarbon and a polyether group selected from the group consisting of polyethylene glycol, polypropylene glycol, and mixtures therof,
wherein more branched surfactant comprising silicone, hydrocarbon and glycerol group(s) is present in the emulsion than surfactant comprising silicone and glycerol group(s) but which lacks hydrocarbon groups.

2. The emulsion of claim 1, wherein the branched surfactant comprising silicone, hydrocarbon and glycerol group(s) is lauryl polyglyceryl-3 polydimethylsiloxyethyl.

3. The emulsion of claim 2, wherein the branched surfactant comprising silicone and glycerol group(s) but which lacks hydrocarbon groups is polyglyceryl-3 disiloxane dimethicone.

4. The emulsion of claim 3, wherein the branched surfactant comprising silicone, hydrocarbon and glycerol group(s) is lauryl PEG-9 polydimethylsiloxy dimethicone.

5. The emulsion of claim 1, wherein the emulsion is an oil-in-water emulsion.

6. The emulsion of claim 5, wherein the branched surfactant comprising silicone, hydrocarbon and glycerol group(s) is lauryl polyglyceryl-3 polydimethylsiloxyethyl.

7. The emulsion of claim 6, wherein the branched surfactant comprising silicone and glycerol group(s) but which lacks hydrocarbon groups is polyglyceryl-3 disiloxane dimethicone.

8. The emulsion of claim 7, wherein the branched surfactant comprising silicone, hydrocarbon and glycerol group(s) is lauryl PEG-9 polydimethylsiloxy dimethicone.

9. The emulsion of claim 1, wherein the weight ratio of branched surfactant comprising silicone, hydrocarbon and glycerol group(s) to surfactant comprising silicone and glycerol group(s) but which lacks hydrocarbon groups present in the emulsion is at least 2:1.

10. The emulsion of claim 5, wherein the weight ratio of branched surfactant comprising silicone, hydrocarbon and glycerol group(s) to surfactant comprising silicone and glycerol group(s) but which lacks hydrocarbon groups present in the emulsion is at least 2:1.

11. The emulsion of claim 1, further comprising at least one wax.

12. The emulsion of claim 5, further comprising at least one wax.

13. The emulsion of claim 11, wherein wax is present in the emulsion in an amount of at least 10% by weight of the total weight of the emulsion.

14. The emulsion of claim 12, wherein wax is present in the emulsion in an amount of at least 10% by weight of the total weight of the emulsion.

15. The emulsion of claim 1, further comprising at least one coloring agent.

16. The emulsion of claim 5, further comprising at least one coloring agent.

17. The emulsion of claim 1, in the form of a mascara.

18. The emulsion of claim 5, in the form of a mascara.

19. A method of enhancing the appearance of eyelashes comprising applying to the eyelashes the emulsion of claim 1.

20. A method of improving adhesion to and/or distribution on eyelashes of an emulsion for application to eyelashes comprising adding at least one surfactant comprising silicone, hydrocarbon and glycerol group(s) to the composition.
